(19) United States
(12) Patent Application Publication
(10) Pub. No.: US 2019/0308925 A1
(43) Pub. Date: Oct. 10, 2019

(54) PROCESS FOR THE PREPARATION OF DIALKYL TEREPHTHALATES FROM RECYCLE FEEDSTOCKS

(71) Applicant: SABIC GLOBAL TECHNOLOGIES B.V., BERGEN OP ZOOM (NL)

(72) Inventors: Prashant Patil, Bangalore (IN); Venkata Ramanarayanan Ganapathy Bhotla, Karnataka (IN); Ritesh Nandy, Bangalore (IN)

(21) Appl. No.: 16/308,954

(22) PCT Filed: Jun. 23, 2017
(86) PCT No.: PCT/IB2017/053775

§ 371 Date: Dec. 11, 2018

Related U.S. Application Data
(60) Provisional application No. 62/355,103, filed on Jun. 27, 2016.

(51) Int. Cl. C07C 67/58; C07C 67/56; C07C 67/03; C07C 69/82; C08K 5/12

(52) U.S. Cl. CPC C07C 67/58; C07C 67/56; C08K 5/12; C07C 69/82; C07C 67/03

(57) ABSTRACT

A process for the preparation of a di(C₆₋₂₁ alkyl) phthalate combining a recycle poly((C₁₋₄ alkyl) terephthalate), a molar excess of a C₆₋₂₁ alcohol relative to the poly(C₁₋₄ alkyl) terephthalate), and a catalyst comprising sulfuric acid, a C₁₋₃₀ sulfonic acid, a tetra(C₁₋₆ alkyl) titanate, or a combination comprising at least one of the foregoing, to provide a reaction mixture; and heating the reaction mixture at a temperature greater than 130° C., under conditions effective to provide a product mixture comprising the di(C₇₋₁₃ alkyl) terephthalate.

### BACKGROUND

Dialkyl arylates are used as plasticizers to increase the flexibility, distensibility, and workability of polymers such as polyvinyl chloride (PVC). Dialkyl phthalates of particular interest include di-2-ethylhexyl phthalate (DEHP) (also known as bis(2-ethylhexyl) phthalate) and “diisooctyl phthalate” (DOP) in the plasticizer industry). Dialkyl terephthalates of particular interest include di-2-ethylhexyl terephthalate (DEHT); also known as 1,4-benzenedicarboxylic acid, di(2-ethylhexyl) ester or as “diisooctyl terephthalate” (DOTP) in the plasticizer industry. DOP and DOTP can be used as a plasticizer in a wide variety of applications, such as coating compositions, sealing compositions, and rubber, particularly PVC articles. Due to regulatory concerns, DOTP (derived from 1,4-benzenedicarboxylic acid) has become an increasingly important plasticizer in place of DOP (which is derived from 1,2-benzenedicarboxylic acid).

As is known in the art, longer-chain dialkyl terephthalates. e.g., di(C₆₋₂₁alkyl) terephthalates such as DOTP, are generally synthesized by transesterification, for example transesterification of dimethyl terephthalate (DMT) with 2-ethylhexanol in the presence of a catalyst. Another route includes the esterification of terephthalic acid or anhydride with 2-ethylhexanol in the presence of a catalyst. However, the starting materials for these procedures are generally derived from virgin feedstocks.

Poly(ethylene terephthalate) (PET) is used extensively in the manufacture of fibers, photographic films, and bottles for soft drinks, whereas poly(butylene terephthalate) (PBT) is used in applications such as door and window hardware, automobile luggage racks and body panels, headlight reflectors, and fiber optic cables. Although efforts have been made to recycle PET and PBT waste, the amount of waste is continually increasing. Chemical recycling (feedstock recycling) chemically decomposes the plastic waste into the original feedstock monomers and converts them back to the polymer or other chemicals. In recent years, several processes for depolymerization of PET or PBT by hydrolysis and alcoholysis have been reported, with different depolymerizing agents and operating conditions including acidic or basic conditions. Reports of synthesizing DOTP from PET or PBT rely on use of a tin catalyst. The presence of residual tin in the DOTP, however, is not desirable, especially for compositions used in medical or food applications.

Accordingly, there remains a continuing need for an improved process for the preparation of di(C₆₋₂₁alkyl) terephthalates such as DOTP that can proceed from recycle feedstocks, in particular PET or PBT.

### BRIEF DESCRIPTION

A process for the preparation of a di(C₆₋₂₁alkyl) terephthalate, comprises combining a recycle poly((C₁₋₄ alkyl) terephthalate), a molar excess of a C₆₋₂₁ alcohol relative to the poly((C₁₋₄ alkyl) terephthalate), and a catalyst comprising sulfuric acid, a C₁₋₃₀ sulfonic acid, a tetra(C₁₋₆ alkyl) titanate, or a combination comprising at least one of the foregoing, to provide a reaction mixture; and heating the reaction mixture at a temperature greater than 130° C., under conditions effective to provide a product mixture comprising the di(C₇₋₁₃alkyl) terephthalate.

A di(C₆₋₂₁alkyl) terephthalate made by the above process is also disclosed.

The above described and other features are exemplified by the following FIGURE and detailed description.

### BRIEF DESCRIPTION OF THE FIGURE

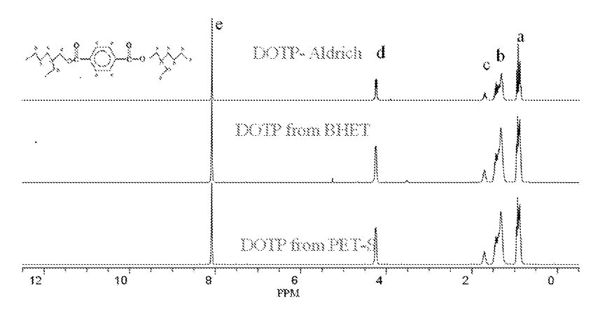

The FIG. 1 is an ¹H NMR (CDCl₃) spectra showing a comparison of NMR spectra of DOTP (Aldrich), DOTP (synthesized from BHET) and DOTP (Synthesized from PET-S).

### DETAILED DESCRIPTION

A process for the preparation of a di(C₆₋₂₁alkyl) terephthalate is disclosed herein. The process does not rely on tin catalysts, and proceeds instead via transesterification of a recycle poly((C₁₋₄ alkyl) terephthalate) with a C₆₋₂₁ alcohol in the presence of a sulfuric acid, a C₁₋₃₀ sulfonic acid, or titanate catalyst. The method is particularly useful for the production of DOTP, and proceeds in high yield.

The starting material is a recycle poly((C₁₋₄ alkyl) terephthalate). As used herein, “recycle” refers to any reclaimed virgin or scrap material, i.e., both pre- and post-consumer waste, and thus includes “virgin” material such as overruns or off-specification material reclaimed during the manufacturing process. Recycle PET or PBT is preferably used. As is understood in the art, poly((C₁₋₄ alkyl) terephthalate)s such as PET or PBT can contain a combination of of terephthalate and isophthalic units, where the mole percent of terephthalic acid units predominate (e.g., are present in an amount of greater than 60 mole percent (mol %), or greater than 75 mol %, or greater than 90 mol %, or greater than 95 mol %), based on the combined terephthalate and isophthalate units. Preferably, a poly((C₁₋₄ alkyl) terephthalate containing greater than 95 mol % of terephthalate units is used, or greater than 98 mol % of terephthalate units, or greater than 99 mol % of terephthalate units. In some embodiments, no detectable amount of a phthalate ester or diester is present, using for example, gas chromatography (GC).

The poly((C₁₋₄ alkyl) terephthalate) is transesterified with a C₆₋₂₁ alcohol. Examples of the alcohol include a saturated or unsaturated, monohydric, straight or branched-chain aliphatic alcohol such as normal- or iso-pentanol, normal- or iso-hexanol, normal- or iso-heptanol, normal- or iso-octanol, 2-ethylhexanol, normal- or iso-nonyl alcohol, normal- or iso-decanol, 2-propyl heptanol, normal- or iso-undecanol, normal or iso-dodecanol, or normal- or iso-tridecanol; or a saturated or unsaturated, monohydric cycloaliphatic alcohol such as cyclohexanemethanol and methylcyclohexanemethanol (including cis and trans isomers, as well as the 1,2-, 1,3-, or 1,4-isomers); or the like; or a combination comprising at least one of the foregoing. Preferably C₆₋₁₃ alcohols are used, and the C₇₋₁₀ alcohols, especially the C₈ alcohols are preferred in the production of the di(C₆₋₂₁alkyl) terephthalates.

In some embodiments, the C₆₋₂₁ alcohol can comprise n-hexanol, cyclohexanol, n-heptanol, 2-ethylhexanol, cyclohexanemethanol, n-octanol, n-nonanol, n-decanol, benzyl alcohol, 2-phenyl ethanol, or a combination comprising at least one of the foregoing. For example, the C₆₋₂₁ alcohol can be 2-ethylhexanol. When the C₆₋₂₁ alcohol is 2-ethylhexanol, the di(C₆₋₂₁alkyl) terephthalate is known as DOTP in the art.

The reaction between the poly((C₁₋₄ alkyl) terephthalate) is conducted in the presence of a catalytic amount of sulfuric acid, a C₁₋₃₀ sulfonic acid, a tetra(C₁₋₆ alkyl) titanate, or a combination comprising at least one of the foregoing.

The sulfuric acid catalyst can be used in the form of concentrated sulfuric acid, i.e., 98% sulfuric acid, or more dilute form. The sulfonic acid catalyst generally comprises straight or branched chain aliphatic sulfonic acids or aromatic sulfonic acids having 1 to 20 carbon atoms, of the formula RSO₃H wherein R is a C₁₋₃₀ hydrocarbyl group, preferably a C₁₋₃₀ alkyl, C₂₋₂₀ alkenyl, C₆₋₃₀ aryl, C₇₋₃₀ arylalkylene (e.g., benzyl), or C₇₋₃₀ alkylarylene (e.g., toluyl). Each of the foregoing groups can optionally be independently substituted with 1 to 3 groups, including halogen, nitrile, nitro, C₁₋₆ alkyl, C₁₋₆ alkoxy, phenyl, or phenoxy. Non-limiting examples of these sulfonic acids include methanesulfonic acid, ethanesulfonic acid, propanesulfonic acid, butane sulfonic acid, naphthalenesulfonic acid, p-toluenesulfonic acid, and dodecylbenzenesulfonic acid. In some embodiments the sulfonic acid is methane sulfonic acid, dodecyl benzene sulfonic acid, para-toluene sulfonic acid, or a combination comprising at least one of the foregoing. The indicated number of carbon atoms in the group includes any substituents. The tetra (C₁₋₆ alkyl) titanate can be a tetra(isopropyl) titanate, also known as titanium isopropoxide titanium tetraisopropoxide (TPT).

In some embodiments, the acid catalyst can be sulfuric acid, methanesulfonic acid, tetraisopropyl titanate, or a combination comprising at least one of the foregoing.

The poly((C₁₋₄ alkyl) terephthalate) and the C₆₋₂₁ alcohol can be present in an amount effective to provide a molar ratio of terephthalate groups to alcohol of 1:2.2 to 1:10, for example 1:3 to 1:6, preferably from 1:3 to 1:4.

The acid catalyst can be present in an amount of 0.1 to 0.5 mol %, or 0.2 to 0.4 mol %, based on the poly((C₁₋₄ alkyl) terephthalate). In an embodiment, the acid catalyst is sulfuric acid, methanesulfonic acid, tetra(isopropyl titanate) or a combination comprising at least one of the foregoing, present in an amount of 0.3 to 0.35 mol %, based on the total moles of the terephthalate units in poly((C₁₋₄ alkyl) terephthalate).

The reaction is initiated by combining the poly((C₁₋₄ alkyl) terephthalate), the C₆₋₂₁ alcohol, and the catalyst to provide a reaction mixture, then heating the reaction mixture to provide a product mixture. Preferably, the reaction mixture is homogenous. The reaction is carried out under conditions effective to provide the di(C₆₋₂₁alkyl) terephthalate, specifically DOTP. A variety of conditions can be suitable for the reaction, depending on the particular poly((C₁₋₄ alkyl) terephthalate), alcohol, desired efficiency, catalyst, and other considerations. For example, the reaction can be carried out at a temperature of 160 to 260° C., for example 200 to 220° C., and a pressure of 0.3 to 10 bar. The reaction is preferably carried out at atmospheric pressure, unless the boiling point of the C₆₋₂₁ alcohol is less than the reaction temperature. In such cases, the reaction is carried out under pressure. Furthermore, the reaction is conducted for a suitable period of time, for example 1 to 8 hours, or for 2 to 4 hours. The reaction can be conducted under an inert atmosphere, e.g., under nitrogen or argon.

In some embodiments, the product mixture can comprise the di(C₆₋₂₁alkyl) terephthalate, specifically DOTP, residual C₆₋₂₁ alcohol, water, a C₁₋₄ alcohol by-product, the corresponding dialkylene glycols (e.g., diethylene glycol), one or more other by-products, or a combination comprising at least one of the foregoing. The reaction can be conducted with the concomitant removal of one or more of these, for example water, the C₁₋₄ alcohol by-product, the corresponding dialkylene glycols, or a combination thereof. Alternatively, water, the C₁₋₄ alcohol by-product, or the corresponding C₂₋₈ dialkylene glycol can be removed separately, or after the transesterification is completed.

The process further comprises isolating the di(C₆₋₂₁alkyl) terephthalate, in particular DOTP, from the product mixture. Isolating the di(C₆₋₂₁alkyl) terephthalate can include a series of process steps including one or more of distillation, acid neutralization, and filtration, which can be conducted in any order. In an embodiment, the product mixture is distilled to remove at least a portion of the residual C₆₋₂₁ alcohol, and optionally water, the C₁₋₄ alcohol by-product, the corresponding dialkylene glycols, or a combination thereof. Distillation can be conducted so as to remove these components sequentially or at the same time. In an embodiment, the distillation is conducted to as to provide the C₆₋₂₁ alcohol in high purity, e.g., the distilled C₆₋₂₁ alcohol has a purity of greater than 98%, or greater than 99%, as determined by gas chromatography.

The acid catalyst in the product mixture can be neutralized. In some embodiments, distilling the reaction mixture can be carried out before neutralizing the acid catalyst. In other embodiments, neutralizing the acid catalyst can be carried out before distilling the reaction mixture. In either embodiment, neutralizing the catalyst can first comprise cooling the product mixture to a temperature of less than 100° C., then adding an aqueous alkaline solution. The amount of aqueous alkaline solution that is added is generally equivalent to the amount of acid present in the reaction mixture. Exemplary bases suitable for use in the aqueous alkaline solution include alkali metal salts, particularly sodium salts such as sodium carbonate, and alkali metal hydroxides such as sodium hydroxide. e.g., aqueous sodium hydroxide.

The distillation and neutralization can produce a first intermediate mixture. In some embodiments, the first intermediate mixture can be further distilled to remove water and a final portion of the residual C₆₋₂₁ alcohol to provide a second intermediate mixture.

The first or the second intermediate mixture can be filtered to provide a filtrate comprising the di(C₆₋₂₁alkyl) terephthalate. In some embodiments, isolating the di(C₆₋₂₁alkyl) terephthalate further comprises treating the filtrate with a decolorizing agent such as activated charcoal, and filtering the treated filtrate, for example using a filter aid, to provide the isolated dialkyl arylate, in particular an isolated DOTP.

The process described herein can provide the di(C₆₋₂₁alkyl) terephthalate, in particular DOTP, with a selectivity of greater than 95%, for example, greater than 98%, for example, greater than 99%. A selectivity of greater than 95% means that the product comprises the di(C₆₋₂₁alkyl) terephthalate, in particular DOTP, and less than 5 weight percent (wt %) of the isophthalate diester based on the weight of the product. Similarly, a selectivity of greater than 98% means that the product comprises the di(C₆₋₂₁alkyl) terephthalate, in particular DOTP, and less than 2 wt % of the isophthalate diester, based on the weight of the product, and a selectivity of greater than 99% means that the product comprises the di(C₆₋₂₁alkyl) terephthalate, in particular DOTP, and less than 1 wt % of the isophthalate ester, based on the weight of the product.

In some embodiments, the conversion of the poly((C₁₋₄alkyl) terephthalate) to the di(C₆₋₂₁alkyl) phthalate, in particular DOTP, can be greater than 85%, for example, greater than 90%, for example, greater than 95%, for example, greater than 98%, for example, greater than 99%, based on the moles of the terephthalate units in the poly((C₁₋₄alkyl) terephthalate). The the poly((C₁₋₄alkyl) terephthalate) product can comprise less than 5 wt %, preferably less than 2 wt %, more preferably less than 1 wt % of the corresponding isophthalate diester, based on the weight of the product.

The di(C₆₋₂₁alkyl) terephthalate, in particular the DOTP prepared according to the above-described method can have a color of less than 35, for example greater than 0 to less than 35, or 1 to 34, or 5 to 32, or 10 to 32, or 20 to 32, as determined according to ASTM D1209.

In a specific embodiment, a process for the preparation of DOTP comprises combining a recycle PET. PBT, or a combination thereof, a molar excess of the 2-ethylhexanol, and a catalyst comprising or consisting of sulfuric acid, a C₁₋₃₀ sulfonic acid, a (C₁₋₆ alkyl) titanate, or a combination comprising at least one of the foregoing, to provide a reaction mixture; and heating the reaction mixture at a temperature greater than 130° C., under conditions effective to provide a product mixture comprising the di(2-ethylhexyl) terephthalate. The ratio of moles of the terephthalate units to moles of the 2-ethylhexanol can be 1:2.2 to 1:30, preferably 1:3 to 1:20, and the catalyst can be present in an amount of 0.1 to 1 mol %, based on the moles of the poly(C₁₋₄ alkyl terephthalate). Heating the reaction mixture can be at a temperature of 160 to 260° C., at a pressure of 0.2 to 20 bar, for 1 to 8 hours, preferably 2 to 4 hours, can can further comprise removing water, ethanol or butanol, or other by-products.

The product mixture comprising DOTP can further comprise residual 2-ethylhexanol, water, ethanol, butanol, ethylene glycol, butylene glycol, diethylene glycol, or a combination comprising at least one of the foregoing. Isolating the DOTP can be by distilling the product mixture to remove at least a portion of the residual 2-ethylhexanol from the product mixture; and neutralizing the catalyst in the product mixture. In an embodiment, distillation is conducted to provide 2-ethylhexanol having a purity of greater than 98%, or greater than 99%, as determined by GC. Neutralizing the catalyst can comprise cooling the product mixture to a temperature less than 100° C., then adding an aqueous base. Isolation can further comprise removing any solids from the product mixture, preferably by filtering the product mixture to provide a filtrate comprising the DOTP, preferably wherein removing any solids comprises filtering the distilled, and neutralized the product mixture to provide a filtrate comprising the DOTP. Optionally the product mixture can be treated with activated charcoal. Preferably the distilled and neutralized the product mixture is treated, and more preferably the filtrate is treated with activated charcoal. The DOTP product can comprise less than 5 wt %, preferably less than 2 wt %, more preferably less than 1 wt % of the isophthalate diester, based on the weight of the product. The DOTP product can comprise less than 5 wt %, preferably less than 2 wt %, more preferably less than 1 wt % of the corresponding mono(2-ethyl hexyl) terephthalate isophthalate ester, based on the weight of the product. The yield of the DOTP can be greater than 85%, preferably greater than 90%, more preferably greater than 95%, even more preferably greater than 98%, even more preferably greater than 99%, based on the moles of terephthalate units in the PET or PBT. The DOTP can advantageously have a color of less than 35, determined according to ASTM D1209.

Further described herein are polymer compositions comprising a polymer and the di(C₆₋₂₁alkyl) terephthalate, in particular the di(2-ethylhexyl) terephthalate manufactured as described above. The di(C₆₋₂₁alkyl) terephthalate can be used as a plasticizer in a variety of polymers, particularly PVC, or cellulose acetate-butyrate, cellulose nitrate, polymethyl methacrylate, polystyrene, or polyvinyl butyral. The polymer compositions can be used to manufacture a wide variety of articles, for example beverage closures, sealing materials used in construction joints, and components of medical devices.

The processes of the present disclosure are further illustrated by the following examples, which are non-limiting.

### EXAMPLES

### Examples 1-4

In Examples 1 and 2, a transesterification of BHET with 2-ethyl hexanol in the presence of a catalytic amount of titanium isopropoxide/sulphuric acid was studied in order to establish the feasibility of the reaction in a model experiment.

In Examples 3 and 4, the synthesis of DOTP was carried out by transesterification of scrap PET (PET-S) using the same experimental conditions as in Examples 1 and 2, in order to develop a one-pot synthesis of DOTP directly from PET-S.

Reaction progress was monitored by GC.

### Example 1. DOTP from BHET with Ti Catalyst

First, 25.4 g (0.1 mol) of bis(hydroxyethyl terephthalate) (BHET) was weighed into a 1.0 L three necked round-bottom flask, equipped with an addition funnel, mechanical stirrer and a Dean-Stark apparatus. Next, 2-ethyl-1-hexanol (2-EH) (32.5 g, 0.25 mol), followed by 30 mg of titanium isopropoxide (TPT) (0.3 g/mol of BHET) was added under a nitrogen atmosphere. The reaction mixture was stirred at 210° C. for 4 hours (h) and then terminated by removing the heating and allowing the temperature to lower to 100° C. The Dean-Stark was replaced by a distillation condenser and excess 2-EH was distilled off under vacuum. The reaction flask was further cooled to 90° C. and 0.4 g of NaOH (0.05 mol %, 10 wt % aqueous solution), was added followed by water, to neutralize the catalyst. After 30 minutes (min) stirring, excess dry ice was added to the reaction flask in order to neutralize excess caustic. After that solids were filtered off using Celite bed. To the obtained filtrate 5 wt % of activated charcoal was added, stirred at 60° C. for 30 min, and filtered to provide DOTP as colorless viscous oil.

### Example 2. DOTP from BHET with H₂SO₄ as Catalyst

First, 25.4 g (0.1 mol) of BHET was weighed into a 1.0 L three necked round-bottom flask, equipped with an addition funnel, mechanical stirrer and a Dean-Stark apparatus. To this 2-ethyl-1-hexanol (32.5 g, 0.25 mol), followed by H₂SO₄ catalyst (0.108 g/mol of BHET) was added under a nitrogen atmosphere. The reaction mixture was stirred at 210° C. for 4 h and then terminated by removing the heating and allowing the temperature to decrease to 100° C. The Dean-Stark was replaced by a distillation condenser and excess 2-EH was distilled off using vacuum. The reaction flask was further cooled to 90° C. and 0.4 g of, NaOH (0.05 mol %, 10 wt % aqueous solution), was added followed by water, to neutralize the catalyst. After 30 min stirring, excess dry ice was added to the reaction flask in order to neutralize excess caustic. After that, solids were removed by filtration through a Celite bed. To the obtained filtrate 5 wt % of activated charcoal was added, stirred at 60° C. for 30 min, and filtered to give DOTP as colorless viscous oil.

### Example 3. DOTP from PET-Scrap with Ti Catalyst

First, 92 g (0.5 mole) of PET scrap was weighed into a 2.0 L three necked round-bottom flask, equipped with an addition funnel, mechanical stirrer and a Dean-Stark apparatus. To this 2-ethyl-1-hexanol (390 g, 3.0 mol), followed by 150 mg (0.11 mol % or 0.3 g/mol of PET-scrap) of TPT was added under a nitrogen atmosphere. The reaction mixture was stirred at 210° C. for 4 h and then terminated by removing the heating and allowing the temperature to lower to 100° C. The Dean-Stark was replaced by a distillation condenser and excess 2-EH was distilled off using vacuum. The reaction flask was further cooled to 90° C. and 2 g of NaOH (0.05 mol %, 10 wt % aqueous solution) was added followed by water, to neutralize catalyst.

After 30 min stirring, excess dry ice was added to reaction flask in order to neutralize excess caustic. After that, solids were removed by filtration through a Celite bed. To the obtained filtrate 5 wt % of activated charcoal was added, stirred at 60° C. for 30 min, and filtered to give DOTP as colorless viscous oil.

### Example 3. DOTP from PET-Scrap with H₂SO₄ as Catalyst

First, 92 g (0.5 mole) of PET scrap was weighed into a 2.0 L three necked round-bottom flask, equipped with an addition funnel, mechanical stirrer and a Dean-Stark apparatus. To this 2-ethyl-1-hexanol (390 g, 3.0 mol), followed by 54 mg (0.11 mol % or 0.108 g/mol of PET-scrap) of H₂SO₄ was added under an N2 atmosphere. The reaction mixture was stirred at 220° C. for 5 h and then terminated by removing the heat and allowing the temperature to lower to 100° C. The Dean-Stark was replaced by a distillation condenser and excess 2-EH was distilled off using vacuum. The reaction flask was further cooled to 90° C. and 2 g of NaOH (0.05 mol %, 10 wt % aqueous solution) was added followed by water, to neutralize catalyst. After 30 min stirring, excess dry ice was added to reaction flask in order to neutralize excess caustic. After that, solids were removed by filtration through a Celite bed. To the obtained filtrate 5 wt % of activated charcoal was added, stirred at 60° C. for 30 min and filtered to give DOTP as colorless viscous oil.

### Results

The products obtained from BHET and PET-S were characterized by proton nuclear magnetic resonance spectroscopy (¹H NMR) analysis. ¹H NMR (CDCl₃) spectra were recorded on Bruker 300 MHz spectrometer. The comparison of NMR spectra of DOTP (Aldrich), DOTP (synthesized from BHET) and DOTP (Synthesized from PET-S) is shown in the FIGURE. As can be seen, NMR analysis confirms that the product in all of Examples 1-4 was DOTP.

### Example 5. Analysis of Additional Products

Analysis showed other components present with the DOTP. To understand the impurity profile and nature of these impure compounds, additional baseline experiments were carried out. The results of the experiments indicate that the PET-S contains isophthalic acid (IPA) and diethylene glycol (DEG). It is known that IPA, for example, can be added to bottle grade PET to lower the crystallinity of the PET.

### Example 6. Analysis of Yield

Reactions were carried out at 100 and 500 mmoles scale as described above, and the products were isolated. Yield of the isolated products from PET-scrap using Ti-catalyst or H₂SO₄ was in the range of 93-94%, determined using GC analysis of the product, based on the amount of starting material. Purity of the product DOTP was more than 96 weight percent. The products further contained 2-3 wt % of dioctyl isophthalate (DOIP) isomer.

### Example 7. Solvent Recovery

Recovery data for excess 2-ethyl-1-hexanol showed a 96% recovery, having a purity of greater than 99% as determined by GC analysis.

The processes described herein are further illustrated by the following embodiments, which are non-limiting.

### Embodiment 1

A process for the preparation of a di(C₆₋₂₁alkyl) terephthalate, comprising: combining a recycle poly((C₁₋₄alkyl) terephthalate), a molar excess of a C₆₋₂₁ alcohol relative to the poly((C₁₋₄ alkyl) terephthalate), and a catalyst comprising sulfuric acid, a C₁₋₃₀ sulfonic acid, a tetra(C₁₋₆ alkyl) titanate, or a combination comprising at least one of the foregoing, to provide a reaction mixture; and heating the reaction mixture at a temperature greater than 130° C. under conditions effective to provide a product mixture comprising the di(C₇₋₁₃alkyl) terephthalate.

### Embodiment 2

The process of Embodiment 1, wherein the poly((C₁₋₄alkyl) terephthalate) is a poly(ethylene terephthalate), a poly(butylene terephthalate, or a combination comprising at least one of the foregoing; the C₆₋₂₁ alcohol is a C₆₋₁₃ alcohol, preferably n-hexanol, cyclohexanol, heptanol, 2-ethylhexanol, cyclohexanemethanol, n-octanol, iso-octanol, n-nonanol, iso-nonanol, n-decanol, iso-decanol, benzyl alcohol, 2-phenyl ethanol, or a combination comprising at least one of the foregoing; more preferably wherein the C₆₋₁₃ alcohol is 2-ethylhexanol; and the acid catalyst is sulfuric acid, methanesulfonic acid, or tetra(isopropyl) titanate.

### Embodiment 3

The process of any one or more of the preceding Embodiments, wherein a ratio of moles of the terephthalate units to moles of the C₆₋₂₁ alcohol is 1:2.2 to 1:10, preferably 1:3 to 1:6, more preferably 1:3 to 1:4 and the catalyst is present in an amount of 0.1 to 0.5 mole percent, based on the moles of the poly((C₁₋₄ alkyl) terephthalate).

### Embodiment 4

The process of any of the preceding Embodiments, wherein heating the reaction mixture is at a temperature of 160 to 260° C., at a pressure of 0.2 to 10 bar, and for 1 to 8 hours, preferably 2 to 4 hours.

### Embodiment 5

The process of any one or more of the preceding Embodiments, wherein the product mixture further comprises residual C₆₋₂₁ alcohol, water, a C₁₋₄ alkanol, a C₂₋₈ glycol, a C₂₋₈ dialkylene glycol, or a combination comprising at least one of the foregoing.

### Embodiment 6

The process of any one or more of the preceding Embodiments, further comprising at least one of: distilling the product mixture to remove at least a portion of the residual C₆₋₂₁ alcohol, the C₁₋₄alkanol, the C₂₋₈ glycol, or a combination comprising at least one of the foregoing from the product mixture; neutralizing the catalyst in the product mixture; removing any solids from the product mixture; or treating the product mixture with a decolorizing agent, preferably an activated charcoal.

### Embodiment 7

The process of Embodiment 6, wherein the distilled C₆₋₂₁ alcohol has a purity of greater than 98%, or greater than 99%, as determined by gas chromatography.

### Embodiment 8

The process of Embodiment 6 or 7, wherein neutralizing the catalyst comprises cooling the product mixture to a temperature of less than 100° C., then adding an aqueous alkaline solution.

### Embodiment 9

The process of any one or more of Embodiments 6 to 8, wherein removing any solids comprises filtering the product mixture to provide a filtrate comprising the di(C₆₋₂₁alkyl) terephthalate, preferably wherein removing any solids comprises filtering the distilled and neutralized the product mixture to provide a filtrate comprising the di(C₆₋₂₁alkyl) terephthalate.

### Embodiment 10

The process of any one or more of Embodiments 6 to 9, wherein treating the product mixture with activated charcoal comprises treating the distilled and neutralized the product mixture; preferably wherein treating the product mixture with activated charcoal comprises treating the filtrate.

### Embodiment 11

The process of any one or more of the preceding Embodiments, wherein the di(C₆₋₂₁ alkyl) terephthalate comprises less than 5 wt %, preferably less than 2 wt %, more preferably less than 1 wt % of the isophthalate diester, based on the weight of the di(C₆₋₂₁ alkyl) terephthalate.

### Embodiment 12

The process of any one or more of the preceding Embodiments, wherein the yield of the di(C₆₋₂₁ alkyl) terephthalate is greater than 85%, or greater than 90%, preferably greater than 95%, more preferably greater than 98%, even more preferably greater than 99%, based on the moles of terephthalate units in the poly((C₁₋₄ alkyl) terephthalate).

### Embodiment 13

The process of any one or more of the preceding Embodiments, wherein the di(C₆₋₂₁ alkyl) terephthalate has a color of less than 35, determined according to ASTM D1209.

### Embodiment 14

The process of any one or more of the preceding Embodiments, wherein the recycle poly(C₁₋₄ alkyl terephthalate) is poly(ethylene terephthalate), poly(butylene terephthalate, or a combination comprising at least one of the foregoing; the C₁₋₄ alcohol is 2-ethyl hexanol; and the di(C₆₋₂₁alkyl) terephthalate is di(2-ethylhexyl) terephthalate.

### Embodiment 15

A di(C₆₋₂₁alkyl) terephthalate made by the process of any one or more of Embodiments 1 to 14, or a polymer composition comprising the di(C₆₋₂₁alkyl) terephthalate made by the process of any one or more of Embodiments 1 to 14.

The processes disclosed herein can alternatively comprise, consist of, or consist essentially of, any appropriate components or steps herein disclosed. The processes can additionally, or alternatively, be formulated so as to be devoid, or substantially free, of any components, materials, ingredients, adjuvants, species or process steps used in the prior art compositions or processes or that are otherwise not necessary to the achievement of the function and/or objectives of the present processes.

The endpoints of all ranges directed to the same component or property are inclusive and independently combinable. Disclosure of a narrower range or more specific group in addition to a broader range is not a disclaimer of the broader range or larger group. “Combination” is inclusive of blends, mixtures, alloys, reaction products, and the like. The terms “first,” “second,” and the like, herein do not denote any order, quantity, or importance, but rather are used to denote one element from another. The terms “a” and “an” and “the” herein do not denote a limitation of quantity, and are to be construed to cover both the singular and the plural, unless otherwise indicated herein or clearly contradicted by context. “Or” means “and/or.” Reference throughout the specification to “an embodiment”. “another embodiment”, “some embodiments”, and so forth, means that a particular element described in connection with the embodiment is included in at least one embodiment described herein, and may or may not be present in other embodiments. In addition, it is to be understood that the described elements may be combined in any suitable manner in the various embodiments.

Unless otherwise specified herein, any reference to standards, regulations, testing methods and the like, such as ASTM D1209, refer to the standard, regulation, guidance, or method that is in force at the time of filing of the present application.

All cited patents, patent applications, and other references are incorporated herein by reference in their entirety. However, if a term in the present application contradicts or conflicts with a term in the incorporated reference, the term from the present application takes precedence over the conflicting term from the incorporated reference.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

I/We claim:

1. A process for the preparation of a di(C₆₋₂₁alkyl) terephthalate, comprising:
combining a recycle poly((C₁₋₄ alkyl) terephthalate), a molar excess of a C₆₋₂₁ alcohol relative to the poly((C₁₋₄ alkyl) terephthalate), and a catalyst comprising sulfuric acid, a C₁₋₃₀ sulfonic acid, a tetra(C₁₋₆ alkyl) titanate, or a combination comprising at least one of the foregoing, to provide a reaction mixture; and
heating the reaction mixture at a temperature greater than 130° C., under conditions effective to provide a product mixture comprising the di(C₇₋₁₃alkyl) terephthalate.

2. The process of claim 1, wherein
the poly((C₁₋₄alkyl) terephthalate) is a poly(ethylene terephthalate), a poly(butylene terephthalate, or a combination comprising at least one of the foregoing;
the C₆₋₂₁ alcohol is a C₆₋₁₃ alcohol; and
the acid catalyst is sulfuric acid, methanesulfonic acid, or tetra(isopropyl) titanate.

3. The process claim 1, wherein
a ratio of moles of terephthalate units in the recycle poly((C₁₋₄ alkyl) terephthalate) to moles of the C₆₋₂₁ alcohol is 1:2.2 to 1:10; and
the acid catalyst is present in an amount of 0.1 to 0.5 mole percent, based on the poly((C₁₋₄ alkyl) terephthalate).

4. The process claim 1, wherein heating the reaction mixture is at a temperature of 160 to 260° C., at a pressure of 0.2 to 10 bar, and for 1 to 8 hours.

5. The process of claim 1, wherein the product mixture further comprises residual C₆₋₂₁ alcohol, water, a C₁₋₄ alkanol, a C₂₋₈ glycol, a C₂₋₈ dialkylene glycol, or a combination comprising at least one of the foregoing.

6. The process of claim 1, further comprising at least one of:
distilling the product mixture to remove at least a portion of the residual C₆₋₂₁ alcohol, the C₁₋₄ alkanol, the C₂₋₈ glycol, or a combination comprising at least one of the foregoing from the product mixture;
neutralizing the catalyst in the product mixture;
removing any solids from the product mixture; or
treating the product mixture with a decolorizing agent.

7. The process of claim 6, wherein the distilled C₆₋₂₁ alcohol has a purity of greater than 98%, or greater than 99%, as determined by gas chromatography.

8. The process of claim 6, wherein neutralizing the catalyst comprises cooling the product mixture to a temperature of less than 100° C., then adding an aqueous alkaline solution.

9. The process of claim 6, wherein
removing any solids comprises filtering the product mixture to provide a filtrate comprising the di(C₆₋₂₁alkyl) terephthalate.

10. The process of claim 6, wherein
treating the product mixture with activated charcoal comprises treating the distilled and neutralized the product mixture.

11. The process of claim 1, wherein the di(C₆₋₂₁ alkyl) terephthalate comprises less than 5 wt % of the isophthalate diester, based on the weight of the di(C₆₋₂₁ alkyl) terephthalate.

12. The process of claim 1, wherein the yield of the di(C₆₋₂₁alkyl) terephthalate is greater than 85% based on the moles of terephthalate units in the poly((C₁₋₄ alkyl) terephthalate).

13. The process of claim 1, wherein the di(C₆₋₂₁ alkyl) terephthalate has a color of less than 35, determined according to ASTM D1209.

14. The process of claim 1, wherein
the recycle poly(C₁₋₄ alkyl terephthalate) is poly(ethylene terephthalate), poly(butylene terephthalate, or a combination comprising at least one of the foregoing;
the C₁₋₄ alcohol is 2-ethyl hexanol; and
the di(C₆₋₂₁alkyl) terephthalate is di(2-ethylhexyl) terephthalate.

15. A di(C₆₋₂₁alkyl) terephthalate made by the process of claim 1.

16. The process of claim 2, wherein
the C₆₋₂₁ alcohol is a C₆₋₁₃ alcohol,
a ratio of moles of terephthalate units in the recycle poly((C₁₋₄ alkyl) terephthalate) to moles of the C₆₋₁₃ alcohol is 1:3 to 1:6;
the acid catalyst is present in an amount of 0.1 to 0.5 mole percent, based on the poly((C₁₋₄ alkyl) terephthalate);
heating the reaction mixture is at a temperature of 160 to 260° C., at a pressure of 0.2 to 10 bar, for 1 to 8 hours; and
the product mixture further comprises residual C₆₋₁₃ alcohol, water, a C₁₋₄ alkanol, a C₂₋₈ glycol, a C₂₋₈ dialkylene glycol, or a combination comprising at least one of the foregoing.

17. The process of claim 16, wherein
the recycle poly(C₁₋₄ alkyl terephthalate) is poly(ethylene terephthalate), poly(butylene terephthalate, or a combination comprising at least one of the foregoing;
the C₁₋₄ alcohol is 2-ethyl hexanol; and
the di(C₆₋₂₁alkyl) terephthalate is di(2-ethylhexyl) terephthalate.

18. The process of claim 16, further comprising at least one of:
distilling the product mixture to remove at least a portion of the residual C₆₋₁₃ alcohol, the C₁₋₄ alkanol, the C₂₋₈ glycol, or a combination comprising at least one of the foregoing from the product mixture;
neutralizing the catalyst in the product mixture;
removing any solids from the product mixture; or
treating the product mixture with a decolorizing agent, preferably an activated charcoal.

19. The process of claim 17, wherein
the distilled C₆₋₁₃ alcohol has a purity of greater than 99%, as determined by gas chromatography;
neutralizing the catalyst comprises cooling the product mixture to a temperature of less than 100° C., then adding an aqueous alkaline solution;
removing any solids comprises filtering the product mixture to provide a filtrate comprising the di(C₆₋₂₁ alkyl) terephthalate;
treating the product mixture with activated charcoal comprises treating the distilled and neutralized the product mixture;
the yield of the di(C₆₋₂₁ alkyl) terephthalate is greater than 85%;
the di(C₆₋₂₁ alkyl) terephthalate comprises less than 5 wt %, based on the weight of the di(C₆₋₂₁ alkyl) terephthalate; and
the di(C₆₋₂₁ alkyl) terephthalate has a color of less than 35, determined according to ASTM D1209.
